(19) United States
(12) Patent Application Publication
(10) Pub. No.: US 2015/0353854 A1
(43) Pub. Date: Dec. 10, 2015

(54) SYNTHESIS OF FUELS AND CHEMICALS FROM SUGARS

(71) Applicant: PIONEER ENERGY INC, Lakewood, CO (US)

(72) Inventors: John T Henri, Longmont, CO (US); Jan Zygmunt, Longmont, CO (US); Robert Zubrin, Golden, CO (US)

(73) Assignee: PIONEER ENERGY INC, Lakewood, CO (US)

(21) Appl. No.: 14/300,022

(22) Filed: Jun. 9, 2014

(51) Int. Cl. C10L 1/185; C07C 31/125; C07D 307/46; C07C 1/24; C07C 5/10; C10L 1/16; C10L 1/182; C07D 307/08

(52) U.S. Cl. CPC C10L 1/1855; C10L 1/1824; C07C 31/125; C07D 307/08; C07C 1/24; C07C 5/10; C10L 1/16; C07D 307/46; C10L 2200/04; C10L 2200/0423; C10L 2200/0446; C10L 2200/043

(57) ABSTRACT

Methods to synthesize useful alcohol and aromatic hydrocarbon products from sorbitol derived from sugars are described. Sorbitol is dehydrated to form furans such as 2-acetylfuran which is hydrogenated to form hexanols, ethyl tetrahydrofuran, etc useful as fuels and chemicals for industry and commerce. Sorbitol may also be heated over a dehydrating catalyst to form aromatic hydrocarbons useful as fuels.

### RELATED APPLICATIONS

This application claims the benefit of U.S. Provisional Application No. 61/833,176 filed Jun. 10, 2013 entitled “Synthesis of Fuels and Chemicals from Sugars” which is incorporated herein by reference.

### BACKGROUND OF THE INVENTION

Petroleum is a vital source of fuels for transportation and industrial chemicals that produce polymers, plastics, pharmaceuticals, paints and other important chemicals. However, due to economic, environmental and political factors, new sources besides petroleum are being sought for the production of these materials. Technologies to manufacture industrial commodities such as methanol are mature and used to produce the worlds supply from methane, coal, etc. Ethanol is produced by fermentation and hydration of ethylene produced from petroleum.

There has been increased use of ethanol as a fuel additive and also as a fuel itself. Ethanol does not have a high heat of combustion (30 MJ/kg ethanol vs 45 MJ/kg gasoline) thus giving significantly lower mileage than gasoline. Technology to produce ethanol from cellulose is still in development. Efforts have been made to develop a synthesis of ethanol from synthesis gas but no satisfactory technologies have materialized yet.

Various research efforts are underway to produce butanol that is also an important fuel alternative and industrial chemical by fermentation processes. However the economy of these processes due to the time required for each fermentation cycle, environmental problems from large amounts of water consumption for these processes and difficulty of isolation of butanol from broths along with its toxicity to microbes are challenges that stand in the way.

Thermochemical processes can be far more advantageous over fermentation processes for the production of chemicals. One example is the production of acetic acid, one of the world's most important chemicals which was produced by fermentation is now produced by synthesis. The discovery and development of catalytic carbonylation by homogenous catalysis chemistry now enables the economical and clean production of most of the worlds acetic acid supply from synthesis gas by carbonylation of methanol.

The present invention similarly employs chemistry and avoids the issues mentioned above employing novel methods to synthesize six carbon alcohols like hexanol (39 MJ/kg calorific value) and other products useful as fuels and chemicals from sorbitol obtained from biomass. The production of alcohols, hydrocarbons, ketones and other products by these methods offer the potential for higher energy fuels that are readily compatible with existing automotive and transportation infrastructure.

### BRIEF DESCRIPTION OF DRAWINGS

FIG. 1 describes the synthesis of hexanols from sorbitol.

FIG. 2 describes the synthesis of olefins from sorbitol.

FIG. 3 describes the synthesis of ethyl tetrahydrofuran from sorbitol.

FIG. 4 describes the synthesis of 2-acetylfuran and furan from isosorbide.

FIG. 5 describes the synthesis of aromatic hydrocarbons and lower aliphatic hydrocarbons from isosorbide.

FIG. 6 describes a scheme to synthesize hexanols and tetrahydrofurans starting from sucrose by the use of hydrolysis, dehydration and hydrogenation reactions.

FIG. 7 describes an energy estimation of a mixture of products seen in GCMS analysis of the reaction products from a copper chromite catalyzed hydrogenation of 2-acetyl furan.

FIG. 8 describes an energy estimation of a mixture of products seen in GCMS analysis of the reaction products from a copper zinc oxide catalyzed hydrogenation of 2-acetyl furan.

FIG. 9 describes a continuous flow reactor used in hydrogenation reactions described.

### DETAILED DESCRIPTION OF THE INVENTION

The present invention similarly employs chemistry and avoids the issues mentioned above employing novel methods to synthesize six carbon alcohols like hexanol (39 MJ/kg calorific value) and other products useful as fuels and chemicals from sorbitol obtained from biomass. The production of alcohols, hydrocarbons, ketones and other products by these methods offer the potential for higher energy fuels that are readily compatible with existing automotive and transportation infrastructure.

An important aspect of the present invention is a process to convert sorbitol derived from glucose, fructose, cellulose, starch, etc to higher caloric six carbon alcohols by synthetic thermochemical methods. In one embodiment, sorbitol is converted to six numbered olefins. In another embodiment sorbitol is converted to ethyl tetrahydrofuran or tetrahydrofuran. In one embodiment the tetrahydrofuran is further reacted by pyrolysis to form 3-butene-1-ol which is hydrogenated to form butanol.

The products of the present invention such as isomers of hexanols, alkyl tetrahydrofuran, isomers of hexene, hexanones, furan derivatives and aromatic hydrocarbons may be used as fuels and have high calorific values close to gasoline and higher than ethanol. The products of the present invention may be used individually or as mixtures. The products of the invention may be used as fuels and blended with fuels such as gasoline, diesel or other fossil fuels. The products of the present invention may also be used as solvents and chemicals in industry and commerce.

### Definitions

Unless specifically noted otherwise herein, the definitions of the terms used are standard definitions used in the art. Exemplary embodiments, aspects and variations are illustrated in the figures and drawings, and it is intended that the embodiments, aspects and variations, and the figures and drawings disclosed herein are to be considered illustrative and not limiting.

An “alkyl” group is a straight, branched, saturated or unsaturated, aliphatic group having a chain of carbon atoms, optionally with oxygen, nitrogen or sulfur atoms inserted between the carbon atoms in the chain or as indicated. A (C₁₋C₂₀)alkyl, for example, includes alkyl groups that have a chain of between 1 and 20 carbon atoms, and include, for example, the groups methyl, ethyl, propyl, isopropyl, vinyl, allyl, 1-propenyl, isopropenyl, ethynyl, 1-propynyl, 2-propynyl, 1,3-butadienyl, penta-1,3-dienyl, penta-1,4-dienyl, hexa-1,3-dienyl, hexa-1,3,5-trienyl, and the like. An alkyl group may also be represented, for example, as a —(CR¹R²)ₘ— group where R¹ and R² are independently hydrogen or are independently absent, and for example, m is 1 to 8, and such representation is also intended to cover both saturated and unsaturated alkyl groups.

An alkyl as noted with another group such as an aryl group, represented as “arylalkyl” for example, is intended to be a straight, branched, saturated or unsaturated aliphatic divalent group with the number of atoms indicated in the alkyl group (as in (C₁₋C₂₀)alkyl, for example) and/or aryl group (as in (C₅₋C₁₄)aryl, for example) or when no atoms are indicated means a bond between the aryl and the alkyl group. Nonexclusive examples of such group include benzyl, phenethyl and the like.

An “alkylene” group is a straight, branched, saturated or unsaturated aliphatic divalent group with the number of atoms indicated in the alkyl group; for example, a —(C₁₋C₃)alkylene- or —(C₁₋C₃)alkylenyl-.

A “cyclyl” such as a monocyclyl or polycyclyl group includes monocyclic, or linearly fused, angularly fused or bridged polycycloalkyl, or combinations thereof. Such cyclyl group is intended to include the heterocyclyl analogs. A cyclyl group may be saturated, partially saturated or aromatic.

An alcohol is a compound with an alkyl or cyclic alkyl group bearing a hydroxyl functional group. Examples of alcohols are methanol, ethanol, propanol, isopropanol, butanol (including 1-butanol, 2-butanol, isobutanol, tert-butanol), pentanol (and its isomers including 1-pentanol, 2-pentanol, 3-pentanol, isopentanol, neopentanol, cyclopentanol, etc) and straight chain, branched and cyclic isomers of other higher alcohols such as hexanol, (including 1-hexanol, 2-hexanol, 3-hexanol, iso-hexanol, 2-methylpentanol and other isomers), cyclohexanol, methylcyclohexanol, heptanol and nonanol, etc. A higher alcohol is an alcohol having two or more carbons.

Sorbitol is an acyclic six carbon compound bearing six hydroxyl groups, one on each of its six carbons including any of its diastereomers. For example, D-Sorbitol or any of its diastereoisomers may be synthesized by reduction of six carbon sugars like glucose, fructose, mannose or like sugar. Some examples of a sorbitol are glucitol, mannitol,

“Halogen” or “halo” means fluorine, chlorine, bromine or iodine.

A “heterocyclyl” or “heterocycle” is a cycloalkyl wherein one or more of the atoms forming the ring is a heteroatom that is a N, O, or S such as furan, pyrrole, thiophene, tetrahydrofuran, etc. Non-exclusive examples of heterocyclyl include furyl, piperidyl, 4-morpholyl, 4-piperazinyl, pyrrolidinyl, 1,4-diazaperhydroepinyl, 1,3-dioxanyl, and the like.

“Substituted or unsubstituted” or “optionally substituted” means that a group such as, for example, alkyl, aryl, heterocyclyl, (C₁-C₈)cycloalkyl, hetrocyclyl(C₁-C₈)alkyl, aryl(C₁-C₈)alkyl, heteroaryl, heteroaryl(C₁-C₈)alkyl, and the like, unless specifically noted otherwise, maybe unsubstituted or maybe substituted by 1, 2 or 3 substitutents selected from the group such as halo, nitro, trifluoromethyl, trifluoromethoxy, methoxy, carboxy, carboxyester, —NH₂, —OH, —SH, —NHCH₃, —N(CH₃)₂, —SMe, cyano and the like.

The methods of the present invention can comprise, consist of, or consist essentially of the essential elements and limitations of the method described herein, as well as any additional or optional ingredients, components, or limitations described herein or otherwise useful in synthetic organic chemistry.

Synthesis gas or syngas is a mixture of varying amounts of carbon monoxide and hydrogen. Syngas maybe produced by the partial oxidation of materials such as methane, liquid hydrocarbons, coal, biomass, etc.

Biomass is material obtained from living or recently living organisms.

In various embodiments, the current invention is directed to novel methods to prepare C5 and C6 alcohols, alkyl tetrahydrofurans, tetrahydrofuran and other products such as olefins, aromatics, alkanes and ketones. In one embodiment, sorbitol undergoes repeated dehydration to first form intermediates such as 1,4-anhydro sorbitol, 2,5-anhydro sorbitol and 1,5-anhydrosorbitol which are further dehydrated to form intermediates such as isosorbide, 2-acetyl furan, substituted furans, cyclopentanes, etc. The dehydrated products like 2-acetylfuran, substituted furans, substituted cyclopentanones are hydrogenated to form reduced products such as alkyl tetrahydrofuran, alkyl cyclopentanol. The tetrahydrofuran derivatives may be pyrolyzed and reduced to form hexanols.

In one embodiment, sorbitol is dehydrated over a heterogeneous catalyst to form 2-acetyl furan and other substituted furans. In one embodiment, the heterogeneous catalyst is γ-alumina. In one embodiment the catalyst for dehydration is a phosphated γ-alumina. In one embodiment, the heterogeneous catalyst is a zeolite such as ZSM-5. In another embodiment, the catalyst for formation of acetyl furan is a metal oxide such as titania, magnesia, silica, iron oxide, zirconia, lanthanum oxide or a mixture of the above.

In one embodiment, 2-acetyl furan is hydrogenated to form ethyl tetrahydrofuran which is used as a fuel. In another embodiment, 2-acetylfuran is hydrolyzed and hydrogenated to form tetrahydrofuran which is used as a fuel, chemical or solvent or used to make other chemicals such as butanol. In one embodiment tetrahydrofuran is pyrolyzed to form 1,3-butadiene.

In one embodiment, the hydrogenation of substituted furans and cyclopentanes is done with a transition metal catalyst. In one embodiment the catalyst is copper-zinc oxide catalyst. In another rembodiment, the catalyst is copper chromite based catalyst. In another rembodiment the catalyst is a nickel catalyst.

In one embodiment, the hydrogenation of 2-acetylfuran is done with a transition metal catalyst. In one embodiment the catalyst is copper-zinc oxide catalyst. In another embodiment, the catalyst is copper chromite based catalyst. In another embodiment the catalyst is a nickel catalyst.

In one embodiment sorbitol is reacted to form isosorbide which can be converted to aromatic hydrocarbons over a heterogeneous catalyst. In one embodiment the catalyst is ZSM-5. In another embodiment the catalyst is γ-alumina. Other catalysts such as zirconia, silica, titania, magnesia and like metal oxides can be used in the conversion.

In one embodiment, sorbitol is dehydrated to form acetyl furan which is hydrogenated to form hexanols. The hexanols are dehydrated to form olefinic hexenes.

Catalysts used in reductions maybe supported or unsupported. A supported catalyst is one in which the active metal or metals are deposited on a support material e.g. by soaking or wetting the support material with a solution, spraying or physical mixing followed by drying, calcination and finally reduction with hydrogen if necessary to produce the active catalyst. Catalyst support materials used frequently are porous solids with high surface areas such as silica, alumina, titania, magnesia, carbon, zirconia, zeolites, etc.

Hydrogenations may also be done using Raney type sponge catalysts such as Raney nickel, copper, cobalt, etc optionally bearing promoters such as iron, molybdenum, chromium, palladium, etc.

In one embodiment the hydrogenation is done with a copper chromite, barium promoted catalyst (62-64% Cr₂CuO₄, 22-24% CuO, 6% BaO, 0-4% Graphite, 1% CrO₃, 1% Cr₂O₃).

In another embodiment a hydrogenation may be done by a copper zinc catalyst. An example of the later is commercially available from Johnson Matthey called the CZ29/2T catalyst.

Pressures required for hydrogenation reactions may be from 10 psia to 1600 psia. In one embodiment the reduction is done at a pressure range of 900 to 1200 psi.

In one embodiment, hydrogenation is done using hydrogen at 300 psi pressure and 300° C.

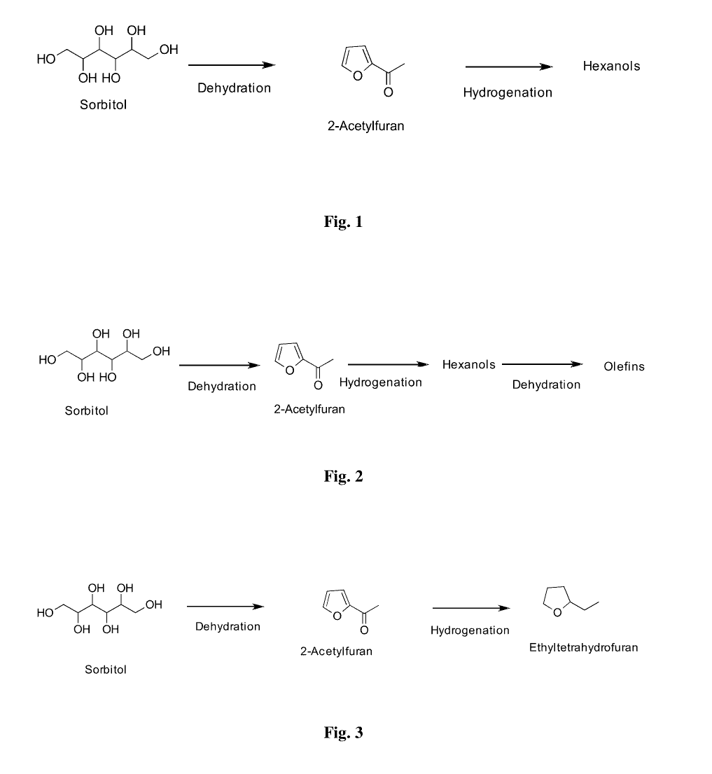

In one variation, compounds of this invention can be synthesized by the steps outlined in FIG. 1. FIG. 1 describes the synthesis of hexanols from sorbitol.

In one variation, compounds of this invention can be synthesized by the steps outlined in FIG. 2. FIG. 2 describes the synthesis of olefins from sorbitol.

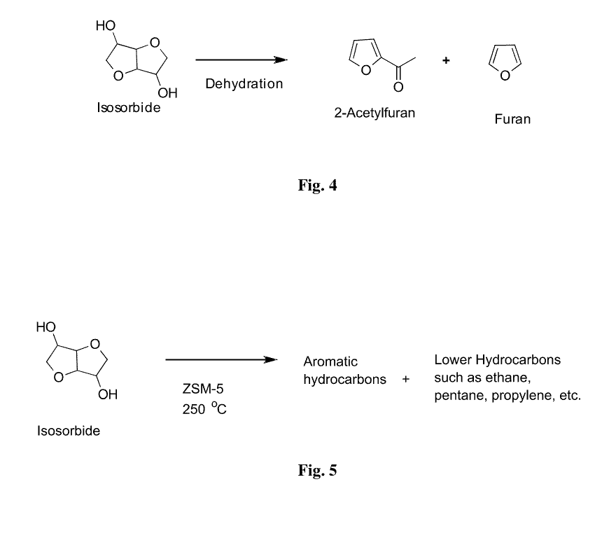

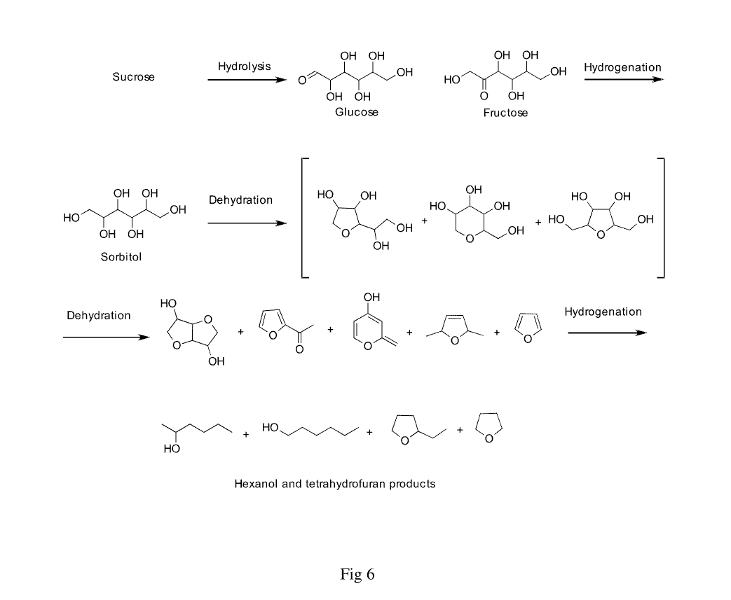

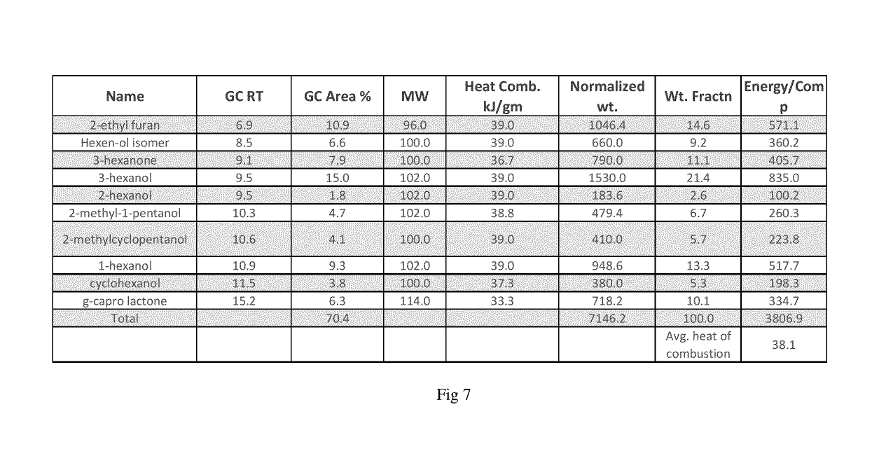

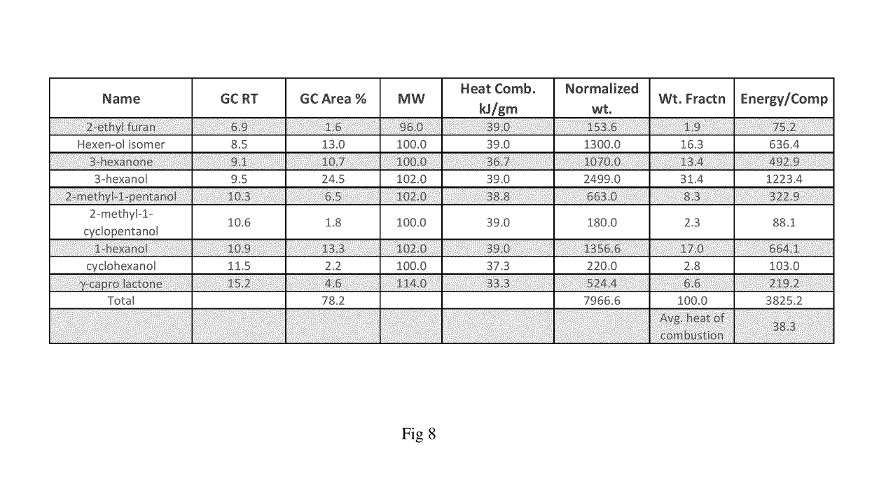

In one variation, compounds of this invention can be synthesized by the steps outlined in FIG. 3. FIG. 3 describes the synthesis of alky tetrahydrofuran from sorbitol.

In one variation, compounds of this invention can be synthesized by the steps outlined in FIG. 4. FIG. 4 describes the synthesis of 2-acetylfuran and furan from isosorbide.

In one variation, compounds of this invention can be synthesized by the steps outlined in FIG. 5. FIG. 5 describes the synthesis of aromatic hydrocarbons and lower aliphatic hydrocarbons from isosorbide.

### EXPERIMENTAL

Although the following experimental procedures are described in detail, they are illustrative and not limitative of the reminder of the description. The starting materials and reagents used in preparing these compounds are either available from commercial suppliers such as Sigma Aldrich, Alfa Aesar, TCI, etc or are prepared by methods well known to a person of ordinary skill in the art, following procedures described in such references as Fieser and Fieser's Reagents for Organic Synthesis, vols. 1-17, John Wiley and Sons, New York, N.Y., 1991; Rodd's Chemistry of Carbon Compounds, vols. 1-5 and supps., Elsevier Science Publishers, 1989; Organic Reactions, vols. 1-40, John Wiley and Sons, New York, N.Y., 1991; March J.: Advanced Organic Chemistry, 4th ed., John Wiley and Sons, New York, N.Y.; and Larock: Comprehensive Organic Transformations, VCH Publishers, New York, 1989.

Standard organic chemical reactions can be achieved by using a number of different reagents, for example, as described in Larock: Comprehensive Organic Transformations, VCH Publishers, New York, 1989.

Samples were analyzed on a Agilent 6890 5973 GCMS system equipped with a JW1 DB624 column with dimensions of 30 m×250μ×1.4μ column. The method ran at 1 ml/min flow, with oven temperature at 40° C. for the first two minutes followed by temperature ramp at 10° C./min to a temperature of 240° C. which was held for 10 minutes. The solvent delay was set at 5 minutes. Chemical identities of obtained alcohols were confirmed by mass spectroscopic analysis on GCMS against a NIST 2011 library as well as by comparison against commercial standards for some compounds.

The presence of sorbitol in the reaction mixtures and liquid products was verified by thin layer chromatography (Silica Gel 60 glass plates, 2.5×7.5 cm, Sigma-Aldrich; eluent—acetone:methanol:water 5:3:1 v/v; visualization was performed by spraying a dry plate with 5% solution of sulfuric acid in ethanol and then heating at 150° C. for 15 min).

Experiment 1: 37.0 g of ZSM-5 catalyst (Tricat, T-25) was mixed with 12.0 g of 17% aqueous solution of isosorbide (99%, Aldrich) and the resulting mixture was loaded into a stainless steel tube (6×1 inch) of the fixed bed reactor. The reactor installed in a good fume hood was sealed, purged with hydrogen and heated to ˜300° C. at an internal pressure of 380 psi. After 30 minutes, the heating was stopped and the catalyst was washed with ethanol (10 mL). GC/MS qualitative analysis of the ethanolic wash showed presence of the mixture of aromatic hydrocarbons including toluene (3.7%), 1.2-dimethylbenzene (11.6%), trimethylbenzenes (17.8%), 2-methylnaphtalene (7.1%), higher alkyl substituted naphatalenes (8.3%), benzofuran (3.1%) and 1-H indenes (5.4%).

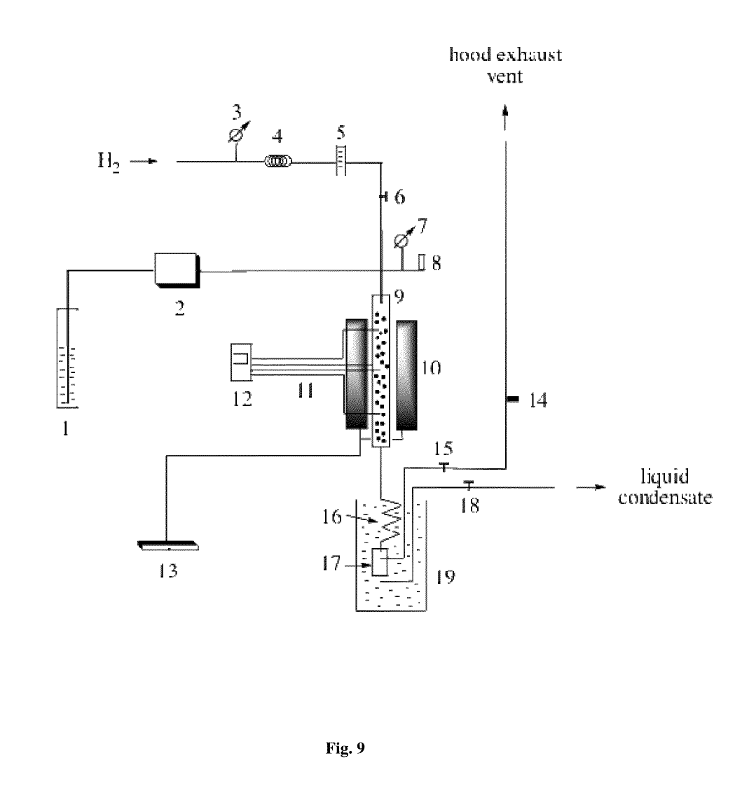

Experiment 2: The continuous flow reactor system (FIG. 9) was installed inside of a good working fume hood. The reactor consisted of a 1″ diameter stainless steel tube 9 bearing four thermocouples 11 connected to the digital reader 12 to measure catalyst bed temperature and reactor outside temperature. The reactor tube 9 (made from Swagelok® parts using the required unions, port connectors, crosses, reducers and valves as needed) had a cross on the top connecting to a HPLC pump's 2 inlet tube on its top arm. One of the side arms was connected to a tube with a pressure gauge 7 and pressure relief valve 8. The other arm of the cross was connected to flow meter 5 through a ball valve 6. To control the flow rate of hydrogen, a 5 foot length of fine 0.005″ id tubing 4 connected by additional flexible tubing to a pressure gauge 3 and a hydrogen cylinder was installed. A 1/16″ tube for liquid feed to an Eldex HPLC pump 2 was fed from a graduated cylinder reservoir 1.

The 1/16″ tube entered through the cross at the reactor top and extended down till it was just above the catalyst bed. The bottom arm of the cross union was attached to the top of the 1″ stainless steel reactor tube 9. The tube was wrapped with heating rope 10 and insulation and heating rope was connected to a controller (Variac) 13. The bottom of the reactor is connected to a coil of ⅛″ stainless steel tube 16 that was connected to short length of ½ inch tube with a drain 17 at its bottom. A coiled tube 16 and a drain 17 were cooled in a bath 19 with ice water to trap a liquid condensate. The liquid collecting vent tube coming out of the bottom of the trap had a needle valve 18 so that it could be opened slowly to collect liquid samples for GC/MS analysis. Non-condensing gases were carried out by a vent near of the top of the drain 17 and the vent had a back pressure valve 15 to control reactor pressure and maintain the required flow rate of hydrogen through the reactor which was calibrated for flow rates at different pressures before the reaction. A gas port 14 was installed after the back pressure valve 15 to collect gas samples for GC analysis.

32.0 gms of ZSM-5 catalyst (Tricat, T-25) was loaded into a reactor tube and the reactor was purged three times with hydrogen and hydrogen pressure was set to 100 psi at flow rate of 90 cc/min The reactor was heated to 280° C. and HPLC pump was turned on to pump 50% aqueous solution of isosorbide (99%, Aldrich) at rate of 1.0 cc/min The experiment was run for 85 minutes at temperature gradient from 280° C. to 287° C. (catalyst temperature at the middle of the reactor) and liquid bilayered samples of the reaction mixture (totally 8 heterogeneous samples) were collected for GC-MS analysis. The results of qualitative analysis of the organic phase (of samples L2, L6 and L8) are shown in Table 1.The aqueous phases (of samples L2, L6 and L8) showed mostly the presence of unreacted isosorbide.

TABLE 1
Organic phase products of isosorbide
dehydration experiment 2 with ZSM-5

| | Reactor temp., ° C. | | | |
|---|---|---|---|---|
| | 280 | 279 | 282 | |
| | Process time, min | | | |
| | (sample #) | | | |
| | 20 | 60 | 80 | |
| | (L2) | (L6) | (L8) | |
| compound | GC-MS % area | | | RT, min |
| benzene | 1.9 | 1.8 | 1.0 | 6.17 |
| toluene | 11.1 | 11.4 | 8.7 | 8.36 |
| ethylbenzene | 2.5 | 2.6 | 2.3 | 10.29 |
| p-xylene | 14.3 | 14.8 | 13.4 | 10.46 |
| o-xylene | 4.1 | 4.9 | 4.4 | 10.99 |
| methyl ethyl benzene | 4.5 | 6.3 | 4.3 | 12.22 |
| trimethyl benzene | 2.4 | 6.3 | 2.0 | 12.25 |
| trimethyl benzene | 6.8 | 7.9 | 8.8 | 12.88 |
| 5-methyl-2-furanocarboxaldehyde | 1.4 | 2.0 | 0.8 | 13.04 |
| benzofuran | 1.1 | 0.8 | 0.8 | 13.25 |
| indane | 2.5 | 2.5 | 2.8 | 13.76 |
| ethyl dimethyl benzene | 1.2 | 1.0 | 1.0 | 14.45 |
| methyl propenyl) benzene | 1.5 | 1.3 | 1.4 | 14.51 |
| methyl indane | 2.7 | 2.9 | 3.7 | 15.50 |
| methyl indene | 1.0 | 1.1 | 1.5 | 15.82 |
| dimethyl indane | 1.9 | 1.9 | 2.4 | 16.14 |
| naphtalene | 2.0 | 2.0 | 2.1 | 16.65 |
| dimethyl indene | 1.0 | 0.9 | 1.2 | 17.58 |
| methyl naphtalene | 7.0 | 6.5 | 7.2 | 18.33 |
| isosorbide | 0.2 | 3.2 | 5.5 | 19.16 |
| ethyl naphtalene | 1.4 | 1.2 | 1.4 | 19.70 |
| dimethyl naphtalene | 4.2 | 4.1 | 4.8 | 19.87 |
| trimethyl naphtalene | 1.3 | 1.1 | 1.3 | 21.14 |

Experiment 3: 39.2 g of ZSM-5 catalyst (Tricat, T-25) was loaded into a continuous flow reactor (FIG. 9) described in the experiment above, the reactor was purged three times with hydrogen and hydrogen pressure was set up to 140 psi at flow rate of ˜70 cc/min The reactor was heated to 300° C. and HPLC pump was turned on to pump 50% aqueous solution of isosorbide (99%, Aldrich) at rate of 1.0 mL/min The process was run for 0.8 h at temperature 300°-345° C. (catalyst temperature at the middle of the reactor) and the reactor pressure 140-180 psi. Then the pressure was increased to 220 psi and the process was run for 1 h at temperature 350°-360° C. and the reactor pressure 220-240 psi. The gas and liquid samples of the reaction mixture were collected for analysis. The results of qualitative analysis of organic liquid phases and gas samples are shown in tables 2 and 3 below.

TABLE 2
Organic phase products of isosorbide dehydration
experiment 3 with ZSM-5

| Reactor temp., ° C. | 341 | 345 | 357 | |
|---|---|---|---|---|
| Process time, min | 24 | 64 | 104 | |
| (sample #) | (L2) | (L6) | (L10) | |
| compound | GC-MS % area | | | RT, min |
| ethyl acetate (from solvent) | 1.0 | 1.1 | 1.1 | 5.41 |
| benzene | 4.7 | 5.4 | 4.8 | 6.27 |
| toluene | 7.9 | 8.8 | 8.1 | 8.54 |
| ethylbenzene | 3.2 | 3.2 | 3.2 | 10.46 |
| p-xylene | 9.0 | 10.0 | 9.6 | 10.67 |
| o-xylene | 4.5 | 5.2 | 5.1 | 11.17 |
| methyl ethyl benzene | 3.8 | 3.3 | 3.3 | 12.40 |
| methyl ethyl benzene | 2.3 | 2.0 | 2.1 | 12.45 |
| trimethyl benzene | 1.0 | 1.5 | 1.3 | 12.54 |
| trimethyl benzene | 4.5 | 5.1 | 5.3 | 13.08 |
| indane | 2.3 | 2.3 | 2.5 | 13.95 |
| indene | 0.3 | 0.7 | 1.0 | 14.26 |
| ethyl dimethyl benzene | 1.2 | 0.9 | 1.1 | 14.65 |
| methyl indane | 1.0 | 0.7 | 0.7 | 14.80 |
| p-cresol + methyl indane | 2.7 | 2.6 | 2.9 | 15.72 |
| methyl indene | 0.6 | 1.1 | 1.4 | 16.04 |
| dimethyl indane | 1.2 | 0.7 | 0.8 | 16.38 |
| dimethyl indane | 1.1 | 0.9 | 0.8 | 16.44 |
| naphtalene | 2.9 | 3.1 | 2.9 | 16.89 |
| methyl naphtalene | 5.6 | 5.0 | 4.5 | 18.61 |
| methyl naphtalene | 2.1 | 2.3 | 1.9 | 18.85 |
| ethyl naphtalene | 2.3 | 1.9 | 1.8 | 19.97 |
| dimethyl naphtalene | 5.3 | 4.0 | 3.5 | 20.16 |
| dimethyl naphtalene | 2.3 | 2.1 | 1.8 | 20.35 |
| dimethyl naphtalene | 1.7 | 1.5 | 1.2 | 20.41 |
| trimethyl naphthalene | 2.2 | 1.5 | 1.4 | 21.43 |

TABLE 3
Gas phase products of isosorbide dehydration
experiment 3 with ZSM-5.

| Reactor temp., ° C. | 341 | 346 | 367 | 350 | |
|---|---|---|---|---|---|
| Process time, min | 26 | 46 | 71 | 96 | |
| (sample #) | (G1) | (G2) | (G3) | (G4) | |
| compound | GC-MS % area | | | | RT, min |
| nitrogen | 8.8 | 12.7 | 13.1 | 13.7 | 1.41 |
| carbon dioxide | 22.7 | 21.7 | 22.7 | 21.9 | 1.46 |
| propane + propene | 30.3 | 27.5 | 28.0 | 26.4 | 1.59 |
| iso-butane | 17.6 | 14.9 | 14.1 | 13.1 | 1.78 |
| n-butane + | 10.3 | 11.4 | 10.5 | 5.7 | 1.94 |
| 2-methyl-1-propene | | | | | |
| 2-butene | 0.8 | 1.7 | 1.7 | 2.2 | 2.03 |
| 1-butene | 0.5 | 1.0 | 1.1 | 1.4 | 2.13 |
| 2-methylbutane | 3.7 | 3.0 | 2.6 | 2.5 | 2.51 |
| pentane | 0.7 | 0.7 | 0.7 | 0.8 | 2.79 |
| furan | 0.5 | 1.0 | 1.5 | 3.0 | 3.02 |
| benzene | 2.2 | 2.5 | 2.0 | 2.1 | 6.13 |
| toluene | 1.6 | 1.9 | 1.5 | 1.6 | 8.46 |

Experiment 4: (Argon medium) 35.6 g of ZSM-5 catalyst (Tricat, T-25) was loaded into a continuous flow reactor (FIG. 9) described above, the reactor was purged three times with argon and argon pressure was set up to 220 psi at flow rate of 42 cc/min The reactor was heated to 350° C. and HPLC pump was turned on to pump 50% aqueous solution of isosorbide (99%, Aldrich) at rate of 1.0 mL/min The process was run for 1.7 h at temperature 300°-350° C. (catalyst temperature at the middle of the reactor) and the reactor pressure 220 psi. The gas and liquid samples of the reaction mixture were collected for analysis. The results of qualitative analysis of organic liquid phases and gas samples are shown in tables 4 and 5 below.

TABLE 4
Organic phase products of isosorbide dehydration
experiment 4 with ZSM-5

| Reactor temp., ° C. | 322 | 348 | 340 | |
|---|---|---|---|---|
| Process time, min | 20 | 60 | 100 | |
| (sample #) | (L2) | (L6) | (L10) | |
| compound | GC-MS % area | | | RT, min |
| ethyl acetate (from solvent) | 1.1 | 1.0 | 1.0 | 5.41 |
| benzene | 4.1 | 4.6 | 4.1 | 6.27 |
| toluene | 8.0 | 8.2 | 7.5 | 8.54 |
| ethylbenzene | 3.2 | 3.4 | 3.4 | 10.46 |
| p-xylene | 9.4 | 9.6 | 9.1 | 10.67 |
| o-xylene | 4.6 | 4.9 | 4.7 | 11.17 |
| methyl ethyl benzene | 4.1 | 3.7 | 3.8 | 12.40 |
| methyl ethyl benzene | 2.4 | 2.3 | 2.4 | 12.45 |
| trimethyl benzene | — | 1.2 | 0.9 | 12.54 |
| trimethyl benzene | 4.8 | 5.2 | 5.4 | 13.08 |
| indane | 2.4 | 2.4 | 2.7 | 13.95 |
| diethyl benzene | 1.0 | 0.9 | 0.9 | 14.10 |
| indene | — | 0.6 | 0.8 | 14.26 |
| ethyl dimethyl benzene | 1.3 | 1.2 | 1.3 | 14.65 |
| methyl indane | 1.0 | 0.8 | 0.9 | 14.80 |
| methyl indane | 2.7 | 2.8 | 3.2 | 15.72 |
| methyl indene | — | 1.0 | 1.3 | 16.04 |
| dimethyl indane | 1.2 | 0.9 | 1.1 | 16.38 |
| dimethyl indane | 1.2 | 1.0 | 1.0 | 16.44 |
| naphtalene | 2.7 | 2.7 | 2.5 | 16.89 |
| dimethyl indane | 1.0 | 0.8 | 0.4 | 17.77 |
| methyl naphtalene | 5.6 | 4.8 | 4.4 | 18.61 |
| methyl naphtalene | 1.8 | 1.8 | 1.4 | 18.85 |
| ethyl naphtalene | 2.4 | 1.9 | 1.8 | 19.97 |
| dimethyl naphtalene | 5.1 | 4.1 | 3.8 | 20.16 |
| dimethyl naphtalene | 2.0 | 1.8 | 1.5 | 20.35 |
| dimethyl naphtalene | 1.5 | 1.3 | 1.7 | 20.41 |
| trimethyl naphtalene | 2.3 | 1.7 | 1.7 | 21.43 |

TABLE 5
gas phase products of isosorbide dehydration
experiment 4 with ZSM-5

| Reactor temp., ° C. | 335 | 349 | 358 | |
|---|---|---|---|---|
| Process time, min | 47 | 62 | 80 | |
| (sample #) | (G1) | (G2) | (G3) | |
| compound | GC-MS % area | | | RT, min |
| nitrogen + argon | 33.5 | 24.7 | 28.5 | 1.45 |
| carbon dioxide | 20.3 | 17.5 | 19.0 | 1.49 |
| propane + propene | 21.0 | 21.7 | 21.0 | 1.63 |
| iso-butane | 11.8 | 13.7 | 12.0 | 1.82 |
| n-butane + | 5.8 | 8.3 | 7.7 | 1.98 |
| 2-methyl-1-propene | | | | |
| 2-butene | 0.7 | 1.3 | 1.5 | 2.06 |
| 1-butene | — | 0.9 | 1.0 | 2.13 |
| 2-methylbutane | 2.0 | 2.8 | 2.4 | 2.55 |
| pentane | — | 0.6 | — | 2.79 |
| furan | 1.4 | 2.8 | 3.3 | 3.05 |
| benzene | 1.8 | 2.4 | 1.8 | 6.14 |
| toluene | 1.7 | 2.5 | 1.8 | 8.46 |

Experiment 5: 33.3 g of gamma-alumina catalyst (Tricat, 1/16″ TL) was loaded into a continuous flow reactor (FIG. 9), the reactor was purged three times with argon and argon pressure was set up to 180 psi at flow rate of 80 cc/min The reactor was heated to 240° C. and HPLC pump was turned on to pump 20% aqueous solution of isosorbide (99%, Aldrich) at rate of 0.5 mL/min The process was run for 1.7 h at temperature 240°-265° C. (catalyst temperature at the middle of the reactor) and the reactor pressure 180 psi. The gas and liquid samples of the reaction mixture were collected for GC-MS analysis. The results of qualitative analysis of the liquid samples (L2, L6, L8) and gas samples (G2, G3, G4, G5, G6) are shown in Table 6 and Table 7, respectively.

TABLE 6
Organic phase products of isosorbide dehydration
experiment 4 with γ-alumina catalyst

| Reactor temp., ° C. | 235 | 266 | 253 | |
|---|---|---|---|---|
| Process time, min | 20 | 60 | 80 | |
| (sample #) | (L2) | (L6) | (S8) | |
| compound | GC-MS % area | | | RT, min |
| ethyl acetate (from solvent) | 5.6 | 23.3 | 16.2 | 5.41 |
| acetic acid | 2.1 | 6.7 | 5.7 | 6.20 |
| cyclopentanone | 2.1 | 1.6 | 1.5 | 9.59 |
| 2-cyclopenten-1-one | 3.0 | 5.4 | 5.1 | 10.64 |
| 2-methyl-cyclopenten-1-one | 6.3 | 4.6 | 4.4 | 11.92 |
| 2-acetylfuran | 8.4 | 12.6 | 9.2 | 12.06 |
| 2-methoxymethylfuran | 1.0 | 1.9 | 1.1 | 12.62 |
| 5-methyl-2-furancarboxaldehyde | 1.1 | 1.1 | 0.9 | 13.15 |
| 3-methyl-2-cyclopeneten-1-one | 2.1 | 1.9 | 1.9 | 13.41 |
| dihydro-5-methyl-2(3H)-furanone | 0.5 | — | 1.5 | 13.65 |
| phenol | 0.4 | 0.9 | 1.2 | 14.18 |
| 3-methyl-1,2-cyclopentanedione | 5.7 | 1.0 | 3.7 | 14.31 |
| isosorbide | 52.7 | 27.3 | 37.0 | 19.41 |

TABLE 7
gas phase products of isosorbide dehydration
experiment 4 with γ-alumina

| | Reactor temp., ° C. | | | | | |
|---|---|---|---|---|---|---|
| | 235 | 257 | 268 | 251 | 266 | |
| | Process time, min (sample #) | | | | | |
| | 28 | 43 | 62 | 82 | 103 | |
| | (G2) | (G3) | (G4) | (G5) | (G6) | RT, |
| compound | GC/MS % area | | | | | min |
| nitrogen + argon | 96.4 | 69.8 | 51.7 | 75.2 | 66.5 | 1.45 |
| carbon dioxide | — | 19.6 | 29.0 | 12.7 | 23.2 | 1.48 |
| furan | — | 5.6 | 15.3 | 9.2 | 10.3 | 3.05 |
| 2-methylfuran | — | — | 1.5 | 2.8 | — | 4.80 |
| toluene | 3.6 | 2.5 | 1.6 | — | — | 8.46 |

Experiment 6: The continuous flow reactor tube was filled with 30.5 gms of ZSM-5 catalyst (Trilobe 1/16″ extradites) obtained from Tricat. A solution of sorbitol was prepared by dissolving 25 g in 100 gm water. Then the reactor pressure was set to 180 psi hydrogen with heating started and temperature at 227° C. inside and 239° C. outside. Hydrogen flow was at ˜100 cc/min. The HPLC pump was turned on and the sorbitol solution pumped at 0.4 cc/min Reaction was run for about 170 minutes and gas and liquid samples of the reaction mixture were collected for GC-MS analysis. The results of qualitative analysis of the organic phase of liquid samples (L12, L14) and gas samples (G5, G6, G7) are shown in Table 8 and Table 9, respectively.

TABLE 8
Products seen in an organic phase for ZSM-5 dehydration
of sorbitol GC-MS analysis.

| Reactor temp., ° C. | 259 | 251 | |
|---|---|---|---|
| Process time, min | 140 (L12) | 160 (L14) | |
| (sample #) | | | |
| compound | GC-MS % area | | RT, min |
| ethyl acetate (from solvent) | 16.0 | 2.4 | 5.41 |
| acetic acid | 19.2 | 3.4 | 6.20 |
| propanoic acid | 1.4 | — | 8.09 |
| toluene | 2.2 | 2.7 | 8.51 |
| ethylbenzene | 0.4 | 1.2 | 10.46 |
| dimethylbenzene | 4.5 | 5.7 | 10.64 |
| dimethylbenzene | 0.6 | 1.8 | 11.16 |
| 2-methyl-2-cyclopenten-1-on | 1.0 | 0.4 | 11.91 |
| 2-acetylfuran | 1.9 | 0.3 | 12.05 |
| methyl ethyl benzene | 0.8 | 2.8 | 12.39 |
| trimethyl benzene | 0.9 | 3.2 | 13.07 |
| benzofuran | 5.6 | 4.0 | 13.42 |
| indane | 0.6 | 2.0 | 13.94 |
| methyl propyle benzene | 0.4 | 1.3 | 14.00 |
| phenol | 4.4 | 1.1 | 14.18 |
| indene | 1.0 | 1.1 | 14.28 |
| ethyl dimethyl benzene | 0.4 | 1.6 | 14.65 |
| methyl phenol | 3.5 | 1.3 | 15.21 |
| methylbenzofuran | 2.7 | 3.8 | 15.38 |
| methyl phenol + methyl indane | 2.9 | 3.8 | 15.67 |
| methyl indene | 1.5 | 3.3 | 16.21 |
| (1-methyl-1-butenyl)-benzene | 0.5 | 2.1 | 16.37 |
| dimethyl phenol | 2.1 | 0.9 | 16.64 |
| dimethyl-1H-inedene | 0.5 | 1.1 | 17.81 |
| dimethyl-1-H-indene | 1.0 | 2.4 | 17.95 |
| methyl naphtalene | 1.4 | 2.8 | 18.58 |
| indanol | 1.0 | 0.8 | 19.75 |
| ethyl naphthalene | 0.3 | 1.0 | 19.96 |
| dimethyl naphthalene | 0.5 | 1.3 | 20.14 |
| trimethyl naphtalene | 0.4 | 1.0 | 21.42 |

TABLE 9
Products seen in gas phase of ZSM-5 dehydration of
sorbitol GC-MS analysis.

| Reactor temp., ° C. | 255 | 254 | 246 | |
|---|---|---|---|---|
| Process time, min | 100 (G5) | 110 (G6) | 132 (G7) | |
| (sample #) | | | | |
| compound | GC-MS % area | | | RT, min |
| nitrogen | 30.6 | 28.6 | 35.8 | 1.45 |
| carbon dioxide | 36.9 | 49.0 | 54.3 | 1.48 |
| propene | 1.1 | 2.4 | 3.4 | 1.62 |
| butane | 3.1 | 3.0 | — | 1.82 |
| 2-methyl-1-propene | — | 2.4 | — | 1.95 |
| acetone* | 28.3 | 14.6 | 6.5 | 3.29 |

*from reactor system clean up

Experiment 7: The continuous flow reactor tube was loaded with 31.5 gms of γ-alumina catalyst 1/16″ trilobe extradites obtained from Tricat. A sorbitol solution was prepared by dissolving 25 gms of sorbitol in 100 ml of water. Heating was started with reactor pressure at ˜210 psi and 238 C inside and 245 C outside temperature with hydrogen flow rate at ˜75 ml/min. The reaction was run for about 120 minutes and gas and liquid samples of the reaction mixture were collected for GC-MS analysis. The results of qualitative analysis of the organic phase of liquid samples (L6, L10, L12) and gas samples (G5, G6, G7) are shown in Table 10 and Table 11, respectively.

TABLE 10
Products seen in an organic phase of γ-alumina dehydration
of sorbitol GC-MS analysis

| Reactor temp., ° C. | 242 | 243 | 239 | |
|---|---|---|---|---|
| Process time, min | 60 | 100 | 120 | |
| (sample #) | (L6) | (L10) | (L12) | |
| compound | GC-MS % area | | | RT, min |
| methylfuran | 0.8 | 1.8 | 1.6 | 5.26 |
| ethyl acetate (from solvent) | 30.5 | 23.2 | 18.0 | 5.41 |
| acetic acid | 6.2 | 4.0 | 3.5 | 6.20 |
| cyclopentanone | 1.1 | 1.3 | 0.9 | 9.59 |
| furfural + 2-cyclopenten-1-on | 3.1 | 4.3 | 3.8 | 10.64 |
| 2-methyl-2-cyclopenten-1-on | 3.5 | 3.8 | 2.9 | 11.91 |
| 2-acetylfuran | 12.6 | 13.7 | 13.0 | 12.05 |
| 2-(methoxymethyl)-furan | 4.0 | 4.4 | 5.2 | 12.61 |
| 5-methyl-2-furancarboxaldehyde | 2.1 | 3.1 | 3.3 | 13.14 |
| methyl-2-cyclopenten-1-on | 1.3 | 1.4 | 1.1 | 13.41 |
| dimethyl-2-cyclopenten-1-on | 1.1 | 1.1 | 0.8 | 13.59 |
| methyl dihydrofuranone | 0.8 | 0.9 | 1.0 | 13.64 |
| phenol | 3.8 | 2.2 | 2.6 | 14.18 |
| methyl-cyclopentadione | 2.1 | 4.3 | 4.6 | 14.28 |
| dimethyl 2-cyclopenten-1-on | 1.2 | 1.0 | 0.8 | 14.53 |
| dimethyl hydroxyl-2- | — | 1.0 | 1.2 | 14.67 |
| cyclopenten-1-on | | | | |
| methyl phenol | 2.7 | 1.0 | 1.3 | 15.20 |
| methyl phenol | 1.4 | 1.0 | 1.0 | 15.66 |
| dimethyl phenol | 1.2 | — | 0.6 | 16.63 |
| hydroxyl phenol | — | 1.4 | 1.7 | 18.46 |
| isosorbide | 6.8 | 4.2 | 5.3 | 19.26 |

TABLE 11
Products seen in gas phase of γ-alumina
dehydration of sorbitol GC-MS analysis.

| | Reactor temp., ° C. | | | | | |
|---|---|---|---|---|---|---|
| | 250 | 241 | 244 | 247 | 239 | |
| | Process time, min (sample #) | | | | | |
| | 52 | 70 | 90 | 110 | 120 | |
| | (G3) | (G4) | (G5) | (G6) | (G7) | RT, |
| compound | GC/MS % area | | | | | min |
| nitrogen | 10.3 | 5.0 | 15.3 | 41.9 | 10.7 | 1.44 |
| carbon dioxide | 28.6 | 50.3 | 32.9 | 23.9 | 39.6 | 1.48 |
| propene | — | — | 0.8 | 0.8 | — | 1.61 |
| water | 6.4 | 7.8 | 3.9 | 3.5 | 4.4 | 1.82 |
| 2-methyl-1-propene | — | — | 0.8 | 0.9 | — | 1.94 |
| furan | 11.6 | 12.7 | 14.1 | 9.9 | 23.3 | 3.05 |
| acetone* | 43.1 | 32.1 | 29.5 | 17.5 | 18.3 | 3.29 |
| 2-methylfuran | — | 2.0 | 2.4 | 1.6 | 3.7 | 4.77 |

*from reactor system clean up

Experiment 8: The continuous flow reactor tube was loaded with 32.5 gms of phosphate alumina catalyst 1/16″ extradites. The catalyst was prepared by taking 49 gms of g-alumina 1/16″ extradites obtained from Tricat and adding about 58 ml of 5.7% phosphoric acid to the extradites. After swirling to wet the alumina thoroughly in a 250 ml RB flask, the water was evaporated in an oven overnight at 130 C followed by calcination at 700 C for three hours. The extrudites were cooled to RT and used directly in the sorbitol conversion. A sorbitol solution was prepared by dissolving 25 gms of sorbitol in 100 ml of water. Heating was started with reactor pressure at ˜210 psi and 238 C inside and 245 C outside temperature with hydrogen flow rate at about 75 ml/min The reaction was run for about 120 minutes and gas and liquid samples of the reaction mixture were collected for GC-MS analysis. The results of qualitative analysis of the organic phase of liquid samples (L5, L7, L9) and gas samples (G5, G6, G7) are shown in Table 12 and Table 13, respectively.

TABLE 12
Products seen in an organic phase of phosphate alumina dehydration
of sorbitol GC-MS analysis.

| Reactor temp., ° C. | 247 | 248 | 243 | |
|---|---|---|---|---|
| Process time, min | 50 | 70 | 91 | |
| (sample #) | (L5) | (L7) | (L9) | |
| compound | GC-MS % area | | | RT, min |
| methylfuran | 1.3 | 0.3 | 1.5 | 5.26 |
| ethyl acetate (from solvent) | 12.8 | 10.4 | 3.8 | 5.41 |
| acetic acid | 2.0 | 1.9 | 1.0 | 6.20 |
| ethyl furan | 0.2 | 0.6 | 1.0 | 6.93 |
| dimethyl furan | 0.3 | 0.8 | 1.3 | 7.02 |
| cyclopentanone | 1.0 | 1.0 | 0.6 | 9.59 |
| furfural + 2-cyclopenten-1-on | 3.5 | 3.1 | 2.8 | 10.64 |
| methyl-2-cyclopenten-1-on | 3.5 | 3.6 | 2.4 | 11.91 |
| 2-acetylfuran | 15.6 | 10.8 | 6.5 | 12.06 |
| methyl-2-furancarboxaldehyde | 1.1 | 0.8 | 1.0 | 12.27 |
| 2-(methoxymethyl)-furan | 1.4 | — | 0.8 | 12.61 |
| methyl-2-furancarboxaldehyde | 2.2 | 1.7 | 2.1 | 13.14 |
| methyl-2-cyclopenten-1-on | 1.4 | 2.0 | 1.5 | 13.41 |
| dimethyl-2-cyclopenten-1-on | 1.6 | 1.6 | 1.0 | 13.59 |
| methyl dihydrofuranone | 1.2 | 0.7 | 0.5 | 13.64 |
| phenol | 3.0 | 2.1 | 1.4 | 14.18 |
| methyl-cyclopentadione | 3.2 | 3.8 | 3.7 | 14.28 |
| dimethyl 2-cyclopenten-1-on | 1.1 | 1.0 | 0.6 | 14.53 |
| dimethyl | — | 1.1 | 1.1 | 14.67 |
| hydroxyl-2-cyclopenten-1-on | | | | |
| methyl phenol | 1.7 | 1.4 | 1.0 | 15.20 |
| methyl phenol | 1.2 | 1.1 | 0.8 | 15.66 |
| hydroxyl phenol | 0.6 | 1.0 | 1.6 | 18.46 |
| isosorbide | 4.3 | 1.9 | 3.0 | 19.26 |

TABLE 13
Products seen in gas phase of phosphate
alumina dehydration of sorbitol by GC-MS

| | Reactor temp., ° C. | | | | | | |
|---|---|---|---|---|---|---|---|
| | 245 | 239 | 231 | 247 | 239 | 249 | |
| | Process time, min (sample #) | | | | | | |
| | 23 | 32 | 42 | 51 | 66 | 78 | |
| | (G3) | (G4) | (G5) | (G6) | (G7) | (G8) | RT, |
| compound | GC/MS % area | | | | | | min |
| nitrogen | 24.5 | 14.4 | 14.3 | 13.6 | 14.6 | 17.9 | 1.42 |
| carbon dioxide | 6.9 | 28.5 | 25.2 | 22.7 | 25.0 | 14.8 | 1.46 |
| propene | — | 1.6 | 3.3 | 2.9 | 3.6 | 2.2 | 1.60 |
| water | 28.7 | 7.4 | 5.5 | 5.4 | 4.5 | 12.2 | 1.80 |
| 2-methyl-1-propene | — | 2.0 | 4.8 | 4.3 | 5.0 | 3.1 | 1.93 |
| 1,3-butadiene | — | 0.9 | 1.0 | 1.0 | 0.9 | 0.9 | 2.00 |
| butene | — | — | 1.3 | 0.9 | 1.1 | 0.7 | 2.04 |
| acetaldehyde | — | — | — | 1.0 | 1.0 | 0.8 | 2.07 |
| butene | — | — | 1.3 | 1.1 | 1.2 | 0.7 | 2.14 |
| furan | 3.2 | 28.5 | 27.1 | 29.3 | 27.1 | 25.2 | 3.03 |
| acetone* | 36.7 | 10.7 | 5.5 | 4.9 | 7.1 | 6.5 | 3.28 |
| 1,4-pentadiene | — | 0.8 | 1.1 | 1.0 | 0.7 | 0.6 | 3.33 |
| 1,3-cyclopentadiene | — | 1.1 | 1.4 | 1.7 | 1.4 | 1.68 | 3.56 |
| methylfuran | — | 3.1 | 4.8 | 4.3 | 4.8 | 5.2 | 4.79 |
| methylfuran | — | 1.0 | 1.3 | 1.6 | 1.6 | 1.9 | 5.04 |
| 2-ethylfuran | — | — | — | — | — | 1.0 | 6.83 |
| 2,5-dimethylfuran | — | — | — | — | — | 1.1 | 6.94 |

*from reactor system clean up

Experiment 9: The continuous flow reactor tube was loaded with 38 gms of Ni/alumina catalyst (50% Ni) NIH-1000 obtained from Unicat. The catalyst was reduced overnight with 5% H₂ in nitrogen at about 180 C followed by reduction with hydrogen gas for 2 hours at ˜200 C. A 40% solution of 2-acetylfuran in methanol was prepared. Heating was started with reactor pressure at ˜220 psi and 238 C inside and 271 C outside temperature with hydrogen flow rate at ˜125 ml/min The reaction was run for about 172 minutes and gas and liquid samples of the reaction mixture were collected for GC-MS analysis. The results of qualitative analysis of the organic phase of liquid samples (L5, L9, L11, L13, L15) and gas samples (G2, G4, G5, G6) are shown in Table 14 and Table 15, respectively.

TABLE 14
Products seen in an organic phase of Ni/Al₂O₃
hydrogenation of 2-acetylfuran GC-MS analysis.

| | Reactor temp., ° C. | | | | | |
|---|---|---|---|---|---|---|
| | 123 | 141 | 161 | 160 | 159 | |
| | Process time, min (sample #) | | | | | |
| | 60 | 100 | 120 | 140 | 160 | |
| | (L5) | (L9) | (L11) | (L13) | (L15) | RT |
| compound | GC-MS % area | | | | | min |
| 2-butanone | 3.8 | | | | | 5.38 |
| 2-butanol | 1.8 | | | | | 5.50 |
| 2-ethyl furan | 2 | 4.7 | 7.4 | 7.9 | 7.8 | 6.90 |
| 2-pentanone | 4.7 | 1.9 | | | | 7.20 |
| 3-pentanone | 5.3 | 2.2 | | | | 7.30 |
| hexene-ol | 3.2 | 2 | 2.55 | 2.8 | 2.7 | 8.50 |
| isomer/ethyl THF | | | | | | |
| 3-hexanone | 9.1 | 3.6 | | 1.7 | 2.0 | 9.10 |
| 2-hexanone | 4.4 | 1.7 | | | | 9.30 |
| 3-hexanol | 2.5 | | | | | 9.50 |
| 2-hexanol | 0.9 | | | | | 9.50 |
| 2-metyl-1-pentanol | 2.8 | | | | 1.6 | 10.28 |
| 2-methyl | 2.8 | 1.1 | | | | 10.56 |
| cyclopentanol | | | | | | |
| cyclohexanol | 1.9 | | | | | 11.40 |
| cyclohexanone | 1 | | | | | 11.80 |
| methyl hexanoate | | | | | | 11.60 |
| 2-acetyl furan + | 15 | 18.2 | 35.6 | 32.1 | 30.4 | 12.00 |
| 1-hydroxyethyl | | 6.7 | | | | 12.00 |
| tetrahydrofuran | | | | | | |
| 1-hydroxyethyl | 4 | 8.3 | 14.9 | 13.4 | 11.84 | 12.20 |
| tetrahydrofuran | | | | | | |
| 4,5-hexanediol | 2.8 | | 2.4 | 4.1 | 4.6 | 13.50 |
| 4,5-hexanediol | 2.2 | | 2.2 | 3.3 | 3.6 | 13.60 |

TABLE 15
Products seen in an gas phase of Ni/Al₂O₃ conversion of
2-acetylfuran GC-MS analysis.

| | Reactor temp., | 185 | 186 | 206 | 207 |
|---|---|---|---|---|---|
| | ° C. | | | | |
| | Process time, | 30 | 50 | 75 | 90 |
| | min | | | | |
| RT | Compound | GC-MS peak area | | | |
| min | Sample | G2 | G4 | G5 | G6 |
| 1.4 | methane | | | 24.9 | 20.2 |
| 1.42 | Air | 20.4 | 23.9 | | |
| 1.47 | Ethane | 34 | 17.1 | 25.1 + ethane | 21.4 |
| 1.5 | Carbon dioxide | | | | |
| 1.6 | Propane | 12.7 | 9.1 | 3.6 | 9.5 |
| 1.8 | Water | | 25.1 | 24.7 | 28.5 |
| 1.77 | Dimethyl ether | 2.5 | 1.9 | | 1.5 |
| 1.8 | isobutene | | | | |
| 1.98 | n-butane | 10.3 | 8.1 | | 7.9 |
| 2.1 | 1-butene | 0.4 | | | |
| 2.5 | Isopentane | 1.1 | 0.9 | | |
| 2.8 | Pentane | 10.24 | 8.0 | | 6.2 |
| 4.4 | Hexane | 4.1 | | 2.2 | 2.4 |
| 5.1 | 2-butanone | | | 2.9 | |
| 6.5 | n-heptane | | | 2.2 | |
| 6.8 | Ethyl furan | | | 8.7 | 0.7 |
| 7.2 | 3-pentanone | | | 2.6 | |

Experiment 10: The reduction reaction was performed with the same catalyst used in the previous example that was in reduced state. A 20% solution of 2-acetyl furan in water/methanol 1:1 solution was prepared. Heating was started with reactor pressure at ˜220 psi and 179 C inside and 189 C outside temperature with hydrogen flow rate at about 150 ml/min. The reaction was run for about 80 minutes. The results of qualitative analysis of the organic phase of liquid samples (L3, L5, L7, L9) and gas sample (G5) are shown in Table 16 and Table 17, respectively.

TABLE 16
Products seen in an organic phase of Ni/Al₂O₃ reduction of
2-acetylfuran GC-MS analysis.

| Reactor temp., ° C. | 185 | 186 | 206 | 207 | |
|---|---|---|---|---|---|
| Process time, min | 30 | 50 | 75 | 90 | |
| (sample #) | | | | | |
| | GC-MS % area | | | | |
| | | | | | RT |
| Compound | L3 | L5 | L7 | L9 | min |
| 2-ethyl furan | | | | | 6.9 |
| Hexene-ol isomer/ethyl THF ? | 0.8 | 8 | 8 | 8.5 | 8.5 |
| 3-hexanone | | ~0.5 | | | 9.1 |
| 3-hexanol | | | | 1.5 | 9.5 |
| 2-hexanol | | | | | 9.5 |
| 2-metyl-1-pentanol | | | | | 10.28 |
| 2-methyl cyclopentanol | | | | | 10.56 |
| 1-hexanol | | | | | 10.95 |
| Cyclohexanol | | | | | 11.4 |
| Sorbic acid | | | | | 11.5 |
| Methyl hexanoate | | | | | 11.6 |
| 1-hydroxyethyl tetrahydrofuran | 28.4 | 25.7 | 29.9 | 29.4 | 12.0 |
| 1-hydroxyethyl tetrahydrofuran | 29.5 | 25.9 | 33 | 30 | 12.2 |
| 2-methylcyclohexanol | | | | | 12.4 |
| 2-methylcyclohexanol | | | | | 12.5 |
| 4,5-hexanediol | 2.8 | 4.6 | 4 | 4.1 | 13.5 |
| 4,5-hexanediol | 2.2 | 3.2 | 3.2 | 3.4 | 13.6 |
| γ-caprolactone | | | | | 15.4 |

TABLE 17
Products seen in an gas phase of Ni/Al₂O₃ reduction of
2-acetylfuran GC-MS analysis.

| Reactor temp., ° C. | 207 | |
|---|---|---|
| Process time, min | 90 | |
| (sample #) | | |
| | GC-MS | |
| Compound | peak area | |
| Sample | G5 | RT min |
| methane | 46.3 | 1.4 |
| Carbon dioxide, air | 44.4 | 1.5 |
| Water | 1.9 | 1.8 |
| methanol | 2.5 | 2.1 |
| pentane | 2.7 | 2.7 |

Experiment 11: The reactor was loaded with 110 gms of copper chromite barium promoted catalyst tablets obtained from Strem Chemicals. The catalyst was reduced overnight with 5% H₂ in nitrogen at about 180 C followed by reduction with hydrogen gas for 2 hours at ˜200 C. A 20% solution of 2-acetyl furan in water/methanol 1:1 solution was prepared. Heating was started with reactor pressure at ˜380 psi and 222 C inside and 231 C outside temperature with hydrogen flow rate at about 150 ml/min. The reaction was run for about 190 minutes. The gas and liquid samples of the reaction mixture were collected for analysis. The results of qualitative analysis of the organic phase of liquid samples (L4, L7, L11, L15, L19) and gas samples (G3, G4, G8) are shown in tables 18 and 19 below. About 93 ml of solution containing about 18.6 gms of 2-acetylfuran was reacted over the catalyst. The liquid samples were combined in a separating funnel, 100 ml of brine was added and the mixture shaken for a few minutes and allowed to stand. The organic layer was separated to give 5.46 gms of product. GC analysis indicated it contained ˜3.5 volume % methanol and only a trace of water.

TABLE 18
Products seen in an organic phase of Copper-Chromite
catalyst₃ conversion of 2-acetylfuran GC-MS analysis.

| | Reactor temp., ° C. | | | | | |
|---|---|---|---|---|---|---|
| | 229 | 244 | 263 | 290 | 304 | |
| | Process time, min (sample #) | | | | | |
| | 40 | 70 | 110 | 150 | 190 | |
| | GC-MS % area | | | | | RT |
| Compound | L4 | L7 | L11 | L15 | L19 | min |
| 2-ethyl furan | 22.3 | 22.4 | 20.2 | 17.2 | 10.9 | 6.9 |
| Hexene-ol | 3.5 | 1.4 | 3.4 | 4.4 | 6.6 | 8.5 |
| isomer/ethyl THF ? | | | | | | |
| 3-hexanone | | | 2.8 | 4.3 | 7.9 | 9.1 |
| 3-hexanol | 3.7 | 2.2 | 6.9 | 9.4 | 15 | 9.5 |
| 2-hexanol | 2 | | 1.4 | 1.5 | 1.8 | 9.5 |
| 2-metyl-1-pentanol | | | 2.2 | 2.7 | 4.7 | 10.28 |
| 2-methyl cyclopentanol | 3 | 3.8 | 3.6 | 4.1 | 4.1 | 10.56 |
| 1-hexanol | 2.1 | 1.5 | 2.4 | 3.4 | 9.3 | 10.95 |
| Cyclohexanol | | | 3 | 3.5 | 3.8 | 11.4 |
| Sorbic acid | 9.8 | | 1.3 | | | 11.5 |
| Methyl hexanoate | | | | | | 11.6 |
| 1-hydroxyethyl | 5 | 6.35 | 5.6 | 4.8 | | 12.0 |
| tetrahydrofuran | | | | | | |
| 1-hydroxyethyl | 4 | 5.2 | 5.9 | 5.6 | | 12.2 |
| tetrahydrofuran | | | | | | |
| 2-methylcyclohexanol | | | 1.0 | 1.3 | 1.8 | 12.4 |
| 2-methylcyclohexanol | | | | | 1 | 12.5 |
| 4,5-hexanediol | 8.2 | 8.6 | 5.6 | 3.3 | | 13.5 |
| 4,5-hexanediol | 8.7 | 6.6 | 3.9 | 2.3 | | 13.6 |
| γ-caprolactone | 2.7 | 5.3 | 7.2 | 7.9 | 6.3 | 15.4 |

TABLE 19
Products seen in an gas phase of Copper-Chromite catalyst₃ conversion
of 2-acetylfuran C-MS analysis.

| Reactor temp., ° C. | 261 | 262 | 304 | |
|---|---|---|---|---|
| Process time, min | 85 | 100 | 180 | |
| (sample #) | | | | |
| | GC-MS % area | | | |
| | | | | RT |
| Compound | G3 | G4 | G8 | min |
| Carbon dioxide, methane, nitrogen | 70 | 73.5 | 77 | 1.5 |
| Water | 1.3 | 1.3 | | 1.8 |
| Methanol | 1.5 | 1.6 | 2.2 | 2.15 |
| Pentane | | 3.7 | 1.3 | 2.8 |
| Hexane | 1.5 | | 2.4 | 4.4 |
| 2-ethyl furan | 14.8 | 22.4 | 15.9 | 6.8 |

Experiment 12: The reactor was loaded with 101 gms of copper zinc oxide catalyst tablets obtained from Unicat catalysts. The catalyst was reduced overnight with 5% H2 in nitrogen at about 180 C followed by reduction with hydrogen gas for 4 hours at ˜180 C. A 20% solution of 2-acetyl furan in water/methanol 1:1 solution was prepared. Heating was started with reactor pressure at ˜360 psi and 184 C inside and 195 C outside temperature with hydrogen flow rate at about 150 ml/min. The reaction was run for about 180 minutes. The gas and liquid samples of the reaction mixture were collected for analysis. The results of qualitative analysis of liquid samples (L3, L7, L11, L16) and gas samples (G3, G8, G11) are shown in tables 20 and 21 below. The liquid samples were combined in a separating funnel, 100 ml of brine was added and the mixture shaken for a few minutes and allowed to stand. The organic layer was separated to give 6.43 gms of product. GC analysis indicated it contained ˜3.4 volume % methanol and a trace of water.

The results of qualitative analysis of the organic phase of liquid samples (L3, L7, L11, L16) and gas samples (G3, G8, G11) are shown in Table 20 and Table 21, respectively.

TABLE 20
Products seen in organic phase of Copper-Zinc Oxide catalyst₃ conversion
of 2-acetylfuran GC-MS analysis.

| Reactor temp., ° C. | 221 | 232 | 260 | 305 | |
|---|---|---|---|---|---|
| Process time, min | 50 | 90 | 130 | 180 | |
| (sample #) | | | | | |
| | GC-MS % area | | | | |
| | | | | | RT |
| Compound | L3 | L7 | L11 | L16 | min |
| 2-ethyl furan | 29.5 | 25.8 | 17.5 | 1.5 | 6.9 |
| Hexene-ol | 11.2 | 4 | 5.5 | 13 | 8.5 |
| isomer/ethyltetrahydrofuran? | | | | | |
| 3-hexanone | 4.3 | 3 | 4.1 | 10.7 | 9.1 |
| 3-hexanol | 13.5 | 8 | 11.9 | 24.5 | 9.5 |
| 2-hexanol | 3.3 | 2.6 | 2.5 | 2.3 | 9.5 |
| 2-metyl-1-pentanol | | 2.6 | 3.7 | 6.5 | 10.28 |
| 2-methyl cyclopentanol | | 3.3 | 3.1 | 1.8 | 10.56 |
| 1-hexanol | 1.7 | 3.5 | 4.3 | 13.3 | 10.95 |
| Cyclohexanol | | | 2.3 | 2.2 | 11.4 |
| Sorbic acid | 3.5 | | | | 11.5 |
| Methyl hexanoate | | 1.2 | | | 11.6 |
| 1-hydroxyethyl tetrahydrofuran | 5.8 | 6.1 | 5.4 | | 12.0 |
| 1-hydroxyethyl tetrahydrofuran | 5.0 | 5.7 | 5.8 | | 12.2 |
| 4,5-hexanediol | 4.7 | 6.2 | 4.7 | | 13.5 |
| 4,5-hexanediol | 3.6 | 4.5 | 3.2 | | 13.6 |
| γ-caprolactone | | 2.7 | 8.2 | 4.6 | 15.4 |

TABLE 21
Products seen in organic phase of Copper-Zinc Oxide catalyst conversion
of 2-acetylfuran GC-MS analysis.

| Reactor temp., ° C. | 226 | 300 | 304 | |
|---|---|---|---|---|
| Process time, min | 74 | 158 | 176 | |
| (sample #) | | | | |
| | GC-MS % area | | | |
| | | | | RT |
| Compound | G3 | G8 | G11 | min |
| Carbon dioxide, methane, nitrogen | 70 | 75 | 73 | 1.5 |
| Water | 11 | 1.9 | 1.3 | 1.8 |
| Pentane | | 3.7 | | 2.8 |
| Hexane | 3 | 1.5 | 15.8 | 4.4 |
| 2-ethyl furan | 14.9 | 20 | 3.7 | 6.8 |

### REFERENCES

1. Isosorbide as a Renewable Platform chemical for Versatile Applications—Quo Vadis? Marcus Rose et al, Chem. Sus. Chem. 2012, 5, 167-176
2. Renewable Alkanes by Aqueous-Phase Reforming of Biomass-Derived Oxygenates, Huber et al, Angew. Chem. Int. Ed. 2004, 43, 1549-1551
3. The production of carbon materials by hydrothermal carbonization of cellulose; M. Sevilla, CARBON 47 (2009) 2281-2289
4. Hydrotreating Carbohydrates, Sughrue et al U.S. application Ser. No. 12/849,109

The entire disclosures of all documents cited throughout this application are incorporated herein by reference.

What is claimed is:

1. A method to synthesize hexanols comprising the steps of:
a) Dehydrating sorbitol over a catalyst to form 2-acetyl furan, and,
b) Hydrogenating 2-acetyl furan to form hexanols.

2. A method of claim 1 where the sorbitol is obtained from hydrogenation of glucose.

3. A method of claim 1 where the sorbitol is obtained from hydrogenation of fructose.

4. A method of claim 3 where the fructose is obtained by hydrolysis of sucrose.

5. A method of claim 1 where the sorbitol is isolated from a source selected from the group consisting of plant material, biomass or fermentation broth.

6. A method of claim 1 where said hexanols produced are used as a fuel.

7. A method of claim 1 where said catalyst employed in dehydration of sorbitol is selected from a phosphated alumina catalyst, a γ-alumina catalyst, a zeolite or doped zeolite.

8. A method of claim 1 where said catalyst employed in hydrogenation of 2-acetyl furan comprises a transition metal.

9. A method of claim 1 where said catalyst employed in hydrogenation of 2-acetyl furan comprises two or more transition metals.

10. A method of claim 12 where said catalyst employed in hydrogenation2-acetyl furan is a copper chromite barium promoted catalyst.

11. A method of claim 1 where said catalyst employed in hydrogenation of 2-acetyl furan is a copper and zinc based catalyst.

12. A method to synthesize an alkyl tetrahydrofuran comprising the steps of:
c) Dehydrating sorbitol to form 2-acetyl furan, and,
d) Hydrogenating 2-acetyl furan to form said alkyl tetrahydrofuran.

13. A method of claim 12 where the alkyl group is selected from methyl, ethyl and propyl groups.

14. A method of claim 12 where the sorbitol is obtained from hydrogenation of glucose.

15. A method of claim 12 where the sorbitol is obtained from hydrogenation of fructose.

16. A method of claim 15 where the fructose is obtained by hydrolysis of sucrose.

17. A method of claim 12 where the sorbitol is isolated from a source selected from the group consisting of plant material, biomass or fermentation broth.

18. A method of claim 12 where said alkyl tetrahydrofuran product produced is used as a fuel.

19. A method of claim 12 where said catalyst employed in dehydration of sorbitol is selected from a phosphated alumina catalyst, a γ-alumina catalyst, a zeolite or doped zeolite.

20. A method of claim 12 where said catalyst employed in hydrogenation of 2-acetyl furan comprises a transition metal.

21. A method of claim 12 where said catalyst employed in hydrogenation of 2-acetyl furan comprises two or more transition metals.

22. A method of claim 12 where said catalyst employed in said hydrogenation 2-acetyl furan is a copper chromite barium promoted catalyst.

23. A method of claim 15 where said catalyst employed in said hydrogenation of 2-acetyl furan is a copper and zinc based catalyst.

24. A method to synthesize aromatic hydrocarbons comprising:
Heating a solution of sorbitol over a dehydrating catalyst.

25. A method of claim 24 where said catalyst employed in dehydration of sorbitol is selected from a phosphated alumina catalyst, a γ-alumina catalyst, a zeolite or doped zeolite.

26. A method of claim 24 where the sorbitol is dissolved in a solution selected from an aqueous solution, a methanol solution or a water-methanol mixed solvent.

27. A method of claim 25 where the aromatic hydrocarbons are hydrogenated to form a product comprising C-7 to C13 saturated hydrocarbons as major components.

28. A method of claim 37 where the C-7 to C13 hydrocarbons are blended with fuels used as diesel fuel and jet fuel

29. A method to prepare 2-acetyl furan comprising;
heating sorbitol over a dehydration catalyst.

30. A method of claim 29 where said catalyst employed in dehydration of sorbitol is selected from a phosphated alumina catalyst, a γ-alumina catalyst, a zeolite or doped zeolite.

31. A fuel composition comprising a mixture of hexanols and gasoline.

32. A fuel composition comprising a mixture of alkyl tetrahydrofurans and gasoline.
